(19) United States
(12) Patent Application Publication
(10) Pub. No.: US 2024/0090942 A1
(43) Pub. Date: Mar. 21, 2024

(54) SYSTEMS FOR TISSUE DEFECT REPAIR

(71) Applicants: Jan R. Lau, Windsor, CA (US); Christian Spies, San Francisco, CA (US)

(72) Inventors: Jan R. Lau, Windsor, CA (US); Christian Spies, San Francisco, CA (US)

(21) Appl. No.: 18/470,118

(22) Filed: Sep. 19, 2023

Related U.S. Application Data
(60) Provisional application No. 63/376,526, filed on Sep. 21, 2022.
(60) Provisional application No. 63/489,248, filed on Mar. 9, 2023.

(51) Int. Cl. A61B 18/14

(52) U.S. Cl. CPC A61B 18/1492; A61B 2017/00867

(57) ABSTRACT

A system and related methods for repairing a tissue defect having a locating or centering member configured to extend into the tissue defect and to locate one or more components of the system relative to the tissue defect. The locating member can define a non-circular outside shape.

### CROSS-REFERENCE TO RELATED APPLICATION(S)

This application claims the benefit of U.S. Provisional Application No. 63/376,526, filed Sep. 21, 2022, and U.S. Provisional Application No. 63/489,248, filed Mar. 9, 2023, each of which are hereby incorporated by reference herein in its entirety.

### BACKGROUND

### Field

The present disclosure relates to tissue defect repair. Some embodiments are directed toward a system for repair of an atrial septal defect.

### Description of the Related Art

Openings or defects in anatomical tissue can require or benefit from reduction in size or closure. Existing systems often utilize a device to occlude the opening or defect. However, such systems can be relatively large covering a significant surface area around the opening or defect.

### SUMMARY

The systems, methods and devices described herein have innovative aspects, no single one of which is indispensable or solely responsible for their desirable attributes. Without limiting the scope of the claims, some of the advantageous features will now be summarized.

An aspect of the present disclosure involves a system for repairing a tissue defect having a locating member configured to extend into the tissue defect and to locate one or more components of the system relative to the tissue defect. The locating member defines a non-circular outside shape.

In some configurations, the locating member comprises a non-circular balloon.

In some configurations, the balloon is a semi-compliant or compliant balloon.

In some configurations, the locating member comprises at least two balloons.

In some configurations, one or more of the at least two balloons are circular.

In some configurations, the locating member comprises an expandable body, such as a cage or trusswork.

In some configurations, the expandable body comprises a shape memory alloy.

In some configurations, the expandable body is self-expanding.

In some configurations, a first tissue anchor is configured to be implanted into tissue at a first location, and a second tissue anchor is configured to be implanted into tissue at a second location.

In some configurations, the first tissue anchor and the second tissue anchor are the only tissue anchors of the system.

In some configurations, the non-circular outside shape of the locating member defines a major axis. The first tissue anchor and the second tissue anchor are equally spaced on opposite sides of the major axis.

In some configurations, the non-circular outside shape of the locating member defines a minor axis. The first tissue anchor and the second tissue anchor are located on the minor axis.

In some configurations, the system includes a catheter in which the first tissue anchor and the second tissue anchor are disposed, and wherein the locating member is disposed within or beyond a distal end of the catheter.

In some configurations, a first delivery tube comprising a distal end that supports the first tissue anchor, and a second delivery tube comprising a distal end that supports the second tissue anchor.

In some configurations, each of the first delivery tube and the second delivery tube comprise a shape memory alloy.

In some configurations, the distal end of the first delivery tube comprises a curved portion and the distal end of the second delivery tube comprises a curved portion.

In some configurations, the curved portion of the first delivery tube curves in an opposite direction to the curve of the curved portion of the second delivery tube.

In some configurations, the first delivery tube and the second delivery tube are disposed on opposite sides of the centering or locating member.

In some configurations, a first needle is slidably disposed within the first delivery tube and configured to puncture tissue at the first location, and a second needle is slidably disposed within the second delivery tube and configured to puncture tissue at the second location.

In some configurations, each of the first needle and the second needle comprises a conductive material.

In some configurations, the system includes an insulator between each of the first needle and the second needle and a respective one of the first delivery tube and the second delivery tube.

In some configurations, the first tissue anchor and the second tissue anchor each comprise a sharp tip configured to puncture tissue at a respective one of the first location and the second location.

In some configurations, the system includes a first pusher and a second pusher positioned within a respective one of the first delivery tube and the second delivery tube, the first pusher configured to push the first tissue anchor from the first delivery tube and the second pusher configured to push the second tissue anchor from the second delivery tube.

In some configurations, the first pusher and the second pusher are inserted into a tubular body of and push on the sharp tip of a respective one of the first tissue anchor and the second tissue anchor.

In some configurations, each of the first pusher and the second pusher comprises a conductive material.

In some configurations, the system includes an insulator between each of the first pusher and the second pusher and a respective one of the first delivery tube and the second delivery tube.

In some configurations, the system includes a suture coupled to at least one of the first tissue anchor and the second tissue anchor.

In some configurations, the suture is coupled to each of the first tissue anchor and the second tissue anchor.

In some configurations, a suture lock configured to secure a length of a tensioned portion of the suture between the first tissue anchor and the second tissue anchor.

In some configurations, the suture lock comprises a body having a passage through which the suture is passed. A lock screw is supported by the body and is configured to be advanced into the passage and against the suture to secure the body at a location along the suture.

In some configurations, the suture lock further comprises a cutting screw supported by the body and having a sharp end, wherein the cutting screw is configured to be advanced into the passage to cut the suture.

In some configurations, one or both of the lock screw and the cutting screw are moved relative to the body by a driver.

In some configurations, each of the first pusher and the second pusher are hollow.

In some configurations, the defect is an atrial septal defect.

An aspect of the present disclosure involves a system for repairing an atrial septal defect. The system includes a catheter and a centering member slidably supported within the catheter or beyond a distal end of the catheter. The centering member is configured to extend from the catheter into the atrial septal defect. A first delivery tube is slidably supported within the catheter and a first tissue anchor is disposed within a distal end of the first delivery tube. The first delivery tube is configured to be advanced from the catheter to allow implantation of the first tissue anchor into tissue at a first location adjacent a first side of the atrial septal defect. A second delivery tube is slidably supported within the catheter and a second tissue anchor is disposed within a distal end of the second delivery tube. The second delivery tube is configured to be advanced from the catheter to allow implantation of the second tissue anchor into tissue at a second location adjacent a second side of the atrial septal defect.

In some configurations, the first side of the atrial septal defect is opposite to the second side of the atrial septal defect.

In some configurations, the centering member is configured to extend into the atrial septal defect while in a collapsed introduction configuration and expand into an expanded deployed configuration after being positioned within the atrial septal defect.

In some configurations, the centering member comprises a non-circular balloon.

In some configurations, the centering member comprises a substantially ellipsoid balloon.

In some configurations, the centering member comprises at least two balloons.

In some configurations, one or more of the at least two balloons are circular.

In some configurations, the centering member comprises an expandable body, such as a cage or trusswork.

In some configurations, the first location is between about 3 mm to 5 mm from a first edge of the atrial septal defect, and the second location is between about 3 mm to 5 mm from a second edge of the atrial septal defect.

In some configurations, each of the first delivery tube and the second delivery tube comprise a shape memory alloy.

In some configurations, a distal end of the first delivery tube comprises a curved portion and a distal end of the second delivery tube comprises a curved portion.

In some configurations, the curved portion of the first delivery tube and the curved portion of the second delivery tube curve radially outward from a longitudinal axis of the catheter.

In some configurations, the curved portion of the first delivery tube curves in an opposite direction to the curve of the curved portion of the second delivery tube.

In some configurations, the distal end of the first delivery tube and the distal end of the second delivery tube are configured to extend beyond an outer diameter of the catheter.

In some configurations, the first delivery tube and the second delivery tube are disposed on opposite sides of the centering member.

In some configurations, the system includes a first needle slidably disposed within the first delivery tube and configured to puncture septal tissue at the first location, and a second needle slidably disposed within the second delivery tube and configured to puncture septal tissue at the second location.

In some configurations, each of the first needle and the second needle comprises a conductive material.

In some configurations, the system includes an insulator between each of the first needle and the second needle and a respective one of the first delivery tube and the second delivery tube.

In some configurations, the first tissue anchor and the second tissue anchor each comprise a sharp tip configured to puncture tissue at a respective one of the first location and the second location.

In some configurations, the system includes a first pusher and a second pusher positioned within a respective one of the first delivery tube and the second delivery tube, the first pusher configured to push the first tissue anchor from the first delivery tube and the second pusher configured to push the second tissue anchor from the second delivery tube.

In some configurations, the first pusher and the second pusher are inserted into a tubular body of and push on the sharp tip of a respective one of the first tissue anchor and the second tissue anchor.

In some configurations, each of the first pusher and the second pusher comprises a conductive material.

In some configurations, the system includes an insulator between each of the first pusher and the second pusher and a respective one of the first delivery tube and the second delivery tube.

In some configurations, a suture is coupled to at least one of the first tissue anchor and the second tissue anchor.

In some configurations, the suture is coupled to each of the first tissue anchor and the second tissue anchor.

In some configurations, a suture lock is configured to secure a length of a tensioned portion of the suture between the first tissue anchor and the second tissue anchor.

In some configurations, the system includes a suture cutter.

An aspect of the present disclosure involves a system for repairing an atrial septal defect. The system includes a centering member configured to extend into the atrial septal defect. The centering member defines a non-circular outside shape. A first tissue anchor is configured to be implanted into tissue at a first location adjacent a first side of the atrial septal defect and a second tissue anchor configured to be implanted into tissue at a second location adjacent a second side of the atrial septal defect.

In some configurations, the first side of the atrial septal defect is opposite to the second side of the atrial septal defect.

In some configurations, the centering member is configured to extend into the atrial septal defect while in a collapsed introduction configuration and expand into an expanded deployed configuration after being positioned within the atrial septal defect.

In some configurations, the centering member is configured to conform to the shape of the atrial septal defect.

In some configurations, the centering member comprises a non-circular balloon.

In some configurations, the centering member comprises a substantially ellipsoid balloon.

In some configurations, the centering member comprises at least two balloons.

In some configurations, one or more of the at least two balloons are circular.

In some configurations, the centering member comprises an expandable body, such as a cage or trusswork.

In some configurations, the first tissue anchor is configured to be implanted into tissue that is 3 mm to 5 mm from a first edge of the atrial septal defect, and the second tissue anchor is configured to be implanted into tissue that is 3 mm to 5 mm from a second edge of the atrial septal defect.

In some configurations, the system includes a first delivery tube comprising a distal end that supports the first tissue anchor and a second delivery tube comprising a distal end that supports the second tissue anchor.

In some configurations, each of the first delivery tube and the second delivery tube comprise a shape memory alloy.

In some configurations, the distal end of the first delivery tube comprises a curved portion and the distal end of the second delivery tube comprises a curved portion.

In some configurations, the curved portion of the first delivery tube curves in an opposite direction to the curve of the curved portion of the second delivery tube.

In some configurations, the first delivery tube and the second delivery tube are disposed on opposite sides of the centering member.

In some configurations, the system includes a first needle slidably disposed within the first delivery tube and configured to puncture septal tissue at the first location and a second needle slidably disposed within the second delivery tube and configured to puncture septal tissue at the second location.

In some configurations, each of the first needle and the second needle comprises a conductive material.

In some configurations, the system includes an insulator between each of the first needle and the second needle and a respective one of the first delivery tube and the second delivery tube.

In some configurations, the system includes a suture coupled to at least one of the first tissue anchor and the second tissue anchor.

In some configurations, the suture is coupled to each of the first tissue anchor and the second tissue anchor.

In some configurations, a suture lock is configured to secure a length of a tensioned portion of the suture between the first tissue anchor and the second tissue anchor.

In some configurations, the system includes a suture cutter.

An aspect of the present disclosure involves a system for repairing an elongated atrial septal defect. The system includes a centering member configured to extend into the elongated atrial septal defect. The centering member is configured to conform to the shape of the elongated atrial septal defect and to orient one or more components of the system with a lengthwise direction of the elongated atrial septal defect.

In some configurations, a first tissue anchor is configured to be implanted into tissue at a first location adjacent to a first side of the elongated atrial septal defect and proximate a horizontal midline of the elongated atrial septal defect, and a second tissue anchor is configured to be implanted into tissue at a second location adjacent to a second side of the elongated atrial septal defect and proximate the horizontal midline of the elongated atrial septal defect.

In some configurations, the orienting of the centering member locates the first tissue anchor and the second tissue anchor at the first location and the second location, respectively.

An aspect of the present disclosure involves a system for repairing an atrial septal defect that includes a catheter and a centering member slidably supported within the catheter or beyond a distal end of the catheter. The centering member is configured to extend from the catheter into the atrial septal defect. A first tissue anchor is carried by the catheter and configured to be advanced from the catheter to a first location within 3-5 mm of a first side of the atrial septal defect for implantation into tissue. A second tissue anchor is carried by the catheter and configured to be advanced from the catheter to a second location within 3-5 mm of a second side of the atrial septal defect for implantation into tissue.

In some configurations, a first delivery tube slidably is supported within the catheter and configured to carry the first tissue anchor and a second delivery tube is slidably supported within the catheter and configured to carry the second tissue anchor.

In some configurations, a distal end of each of the first delivery tube and the second delivery tube comprises a curved portion.

In some configurations, a distal end of each of the first delivery tube and the second delivery tube is configured to be deflected away from a longitudinal axis of the catheter.

In some configurations, a ramped surface or a curved surface configured to deflect the first delivery tube and the second delivery tube.

An aspect of the present disclosure involves a method for repairing a tissue defect that includes positioning an introducer sheath adjacent a defect of a tissue wall on a first side of the tissue wall. The method can further include positioning a guidewire through the defect of the tissue wall. The method can further include inserting a proximal end of the guidewire into a centering balloon lumen. The method can further include advancing an anchor catheter over the guidewire and within the introducer sheath. The method can further include advancing a balloon member through the anchor catheter to position the balloon member partially through the defect. The method can further include inflating the balloon member. The method can further include advancing a first needle of the anchor catheter through a tissue wall at a first location of the tissue wall, wherein the first location is adjacent to the defect. The method can further include deploying a first delivery tube of the anchor catheter through a first hole created by the first needle. The method can further include deploying a first tissue anchor from the first delivery tube to a second side of the tissue wall.

In some configurations, prior to advancing the first needle through the tissue wall needle at the first location, the method can include deploying the first delivery tube of the anchor catheter and the second delivery tube of the anchor catheter adjacent to the first location of the tissue wall on the first side of the tissue wall, wherein the first delivery tube and the second delivery tube are each configured to curve radially outward from the delivery catheter.

In some configurations, the method can further include energizing the first needle prior to advancing the first needle through the tissue wall.

In some configurations, advancing the first needle of the anchor catheter through the tissue wall at the first location can include advancing each of the first delivery tube and the first tissue anchor through the tissue wall with the first needle. The distal ends of the first needle, the first delivery tube, and the first tissue anchor can form a chamfered edge.

In some configurations, the method can further include advancing a second needle of the anchor catheter through the tissue wall at a second location of the tissue wall, wherein the second location is adjacent to the defect. The method can further include deploying a second delivery tube of the anchor catheter through a second hole created by the second needle. The method can further include deploying a second tissue anchor from the second delivery tube to the second side of the tissue wall.

In some configurations, the method can further include positioning the proximal end of the guidewire within a suture lock catheter. A suture lock can be positioned at a distal end of the suture lock catheter. The method can further include positioning proximal ends of sutures of the first anchor and the second anchor through a passage of the suture lock. The method can further include positioning the suture lock catheter within the introducer sheath and over the guidewire. The method can further include withdrawing the guidewire. The method can further include applying tension to the proximal ends of the sutures. The method can further include locking the tension of the proximal ends of the sutures. The method can further include cutting the proximal ends of the sutures. The method can further include releasing the suture lock from the suture lock catheter.

In some configurations, locking the tension of the proximal ends of the sutures can include advancing a locking screw to capture a portion of each of the sutures against a surface of the passage.

In some configurations, cutting the proximal ends of the sutures can include advancing a cutting screw to cut each of the sutures against a surface of the passage.

In some configurations, releasing the suture lock from the suture lock catheter can include retracting a retaining wire positioned between the suture lock and an inner surface of the suture lock catheter.

An aspect of the present disclosure involves a system for repairing a tissue defect. The system can include a locating member configured to extend into the tissue defect and to locate one or more components of the system relative to the tissue defect, wherein the locating member defines a circular outside shape.

In some configurations, the locating member can include a circular balloon.

In some configurations, the locating member can include at least two balloons.

In some configurations, one or more of the at least two balloons are circular.

In some configurations, the at least two balloons define a circular outside shape.

In some configurations, the locating member can include an expandable body, such as a cage or trusswork.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

FIG. 1 illustrates a system for tissue defect repair implanted in a tissue wall having a defect or opening.

FIG. 2 is a representation of a tissue opening.

FIG. 3 illustrates an anchor delivery catheter.

FIG. 4 is a sectional view of a distal end portion of the anchor delivery catheter of FIG. 3.

FIG. 5 is a distal end view of the anchor delivery catheter FIG. 3.

FIG. 6 is an enlarged view of a portion of the anchor delivery catheter of the dashed line box labeled with the numeral 6 in FIG. 4.

FIG. 7 is a distal end view of an alternative anchor delivery catheter.

FIG. 8A illustrates a first example of a suture lock.

FIG. 8B illustrates a portion of the suture lock of FIG. 8A.

FIG. 9A illustrates a suture lock delivery catheter.

FIG. 9B illustrates a distal end of a suture lock delivery catheter.

FIG. 10A illustrates an example tissue anchor in a collapsed configuration.

FIG. 10B illustrates the tissue anchor of FIG. 10A in an expanded configuration.

FIG. 10C illustrates a distal end view of the tissue anchor of FIG. 10A.

FIG. 10D illustrates the tissue anchor of FIGS. 10A-10C positioned against a tissue defect.

FIG. 10E illustrates a laser cut pattern of the tissue anchor of FIGS. 10A-10D.

FIG. 11A is a side view of an example of a centering balloon positioned within a defect of a septal wall.

FIG. 11B illustrates a side view of an alternative example of a centering balloon positioned within a defect of a septal wall.

FIG. 11C illustrates a side view of yet another alternative example of a centering balloon positioned within a defect of a septal wall.

FIG. 12 is a side view of an example of yet another alternative example of a centering balloon positioned within a defect of a septal wall.

FIG. 13 is an end view of the centering balloon and septal wall defect of FIG. 13, which also shows the tissue anchor locations.

FIG. 14 is a side view of a centering cage positioned within a defect of a septal wall.

FIG. 15 is a sectional view of a distal end portion of a portion of an alternative version of the system in which the centering balloon is external of a catheter of the system.

FIG. 16 is a flow diagram illustrating a method of repairing a tissue defect.

FIG. 17 is a flow diagram illustrating a method of applying a suture lock to one or more sutures.

FIG. 18 is a top, sectional view of the anchor catheter with the delivery tubes deployed from the anchor catheter.

FIG. 19 is a top, sectional view of the anchor catheter with the needles advanced from the delivery tubes through the tissue wall.

FIG. 20 is a top, sectional view of the anchor catheter with the anchor and delivery tube advanced from the anchor catheter through the tissue wall.

FIG. 21 is a top, sectional view of the anchor deployed from the delivery tube on the second side of the tissue wall.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The present disclosure involves a system for repairing a tissue defect or opening. In some configurations, the system is configured for or is well-suited for repairing an atrial septal defect. The atrial septal defect can be naturally occurring, such as a patent foramen ovale (PFO). Alternatively, the atrial septal defect can be the result of a prior procedure, such as an opening resulting from the passage of a catheter through the atrial septum for treatment or replacement of the mitral valve. Intentional creation of a communication between the right and left upper chamber of the heart is referred to as an iatrogenic atrial septal defect (iASD). These iASDs are created to allow access to the left atrium for therapeutic interventions, for example ablation procedures, left atrial appendage closure, or transcatheter mitral valve repair or replacement. Many of these defects are small enough to close spontaneously (e.g., within about 6 months). The larger the defect, the less likely spontaneous closure is to occur, and detrimental effects on the heart can occur.

The field of transcatheter mitral valve replacement is expanding rapidly. It is expected that the first device will be FDA approved in 2024-2025. The delivery systems for those valves are larger than currently used devices. Hence, iASDs will be larger, such that some of them require closure. It is estimated that approximately 10-25% of all iASD created for the purpose of transcatheter mitral valve replacement warrant closure. Currently available transcatheter closure devices rely on large disks placed on each side of the interatrial septum, covering most of the septal surface. This makes repeat transseptal puncture and access to the left atrium technically challenging. Future access to the left atrium, however, is important, as the technology of transcatheter mitral valve replacement is young and adverse events may occur warranting reintervention in the left atrium. Further, bioprosthetic valves have estimated half-lives of 6-8 years, necessitating the need for foreseeable re-interventions. At least some of the embodiments disclosed herein allow for iASD closure and tissue approximation with relatively less or minimal foreign material implanted, such that simple and unobstructed, future trans-septal access may be maintained. In some embodiments, the system utilizes a suture or other tether.

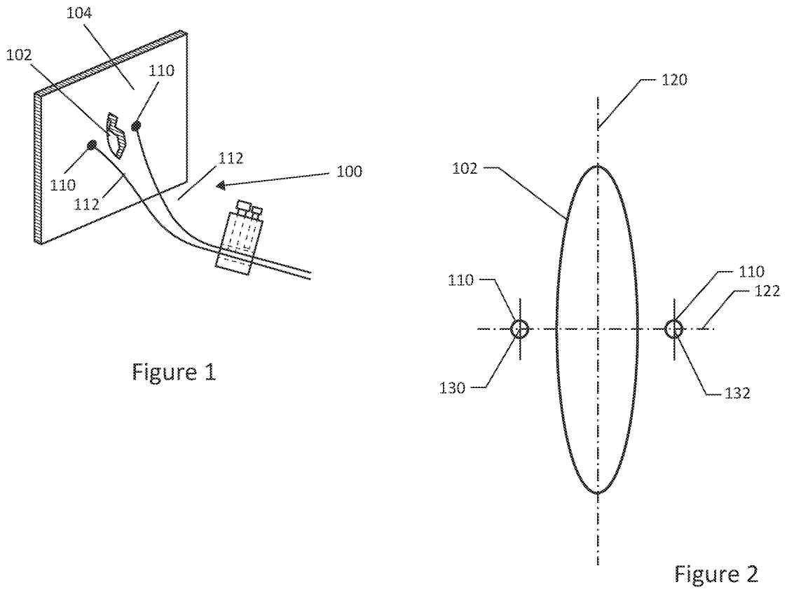

FIG. 1 illustrates a portion of an embodiment of a tissue defect or opening closure system 100 implanted adjacent a tissue defect, which in FIG. 1 is an atrial septal defect or opening 102 in a septal wall 104. The system 100 includes a pair of darts or other tissue anchors 110 implanted in the septal wall 104 on opposing sides of the atrial septal defect 102. One or more tethers, which can be or comprise one or more sutures 112, are coupled to the tissue anchors 110. An effective length of the suture(s) 112 between the tissue anchors 110 can be adjusted to draw the tissue anchors 110 towards one another to reduce a size of the atrial septal defect 102. In some embodiments, the system 100 may be capable of substantially or fully closing the atrial septal defect 102. Closing the atrial septal defect 102 can mean bringing the edges of the tissue defining the atrial septal defect 102 into contact with one another. A suture lock 114 can be employed to maintain a desired effective length of the suture(s) 112. As discussed further below, the tissue anchors 110, suture(s) 112, suture lock 114, and other components of the system 100 can be delivered to the heart, the atrium, and to the septal wall 104 via a catheter using imaging guidance, such as fluoroscopy and/or echocardiography.

FIG. 2 illustrates a simplified form of the atrial septal defect 102. However, the defect 102 of FIG. 2 can represent defects or openings in other tissue. The present inventor has discovered that atrial septal defects 102 in particular and certain other types of tissue openings more generally are often non-circular in shape. Such tissue defects or openings 102 are often elongate in shape, such as generally in the form of an oval or ellipse. FIG. 2 illustrates an oval tissue defect 102 to simplify a discussion of the system 100. However, the concepts discussed in connection with the oval tissue opening 102 of FIG. 2 are equally applicable to an irregular, but substantially oval tissue opening 102 that is perhaps more typical of actual anatomic openings or defects.

The tissue opening 102 of FIG. 2 defines a major axis 120 that extends in a lengthwise direction of the tissue opening 102 or a direction of a maximum dimension of the tissue opening 102. The tissue opening 102 also defines a minor axis 122 that extends in a widthwise direction of the tissue opening 102 or a direction that is perpendicular to the major axis 120. The minor axis 122 can be considered a horizontal midline. The major axis 120 can be considered a vertical midline. The major axis 120 and the minor axis 122 each pass through a center of the tissue opening 102. As described further herein, the system 100 is configured to deliver and/or implant the tissue anchors 110 in a specific orientation relative to the tissue opening 102. In the illustrated arrangement, the system 100 is configured to deliver and/or implant the tissue anchors 110 to a respective one of a first location 130 and a second location 132 on opposite sides of the major axis 120, and therefore on opposite sides of the tissue opening 102. In some configurations, the system 100 is configured to deliver and/or implant the tissue anchors 110 at the first location 130 and the second location 132, each of which are located on or near the minor axis 122. The first location 130 and the second location 132 may be equidistant or substantially equidistant from the major axis 120.

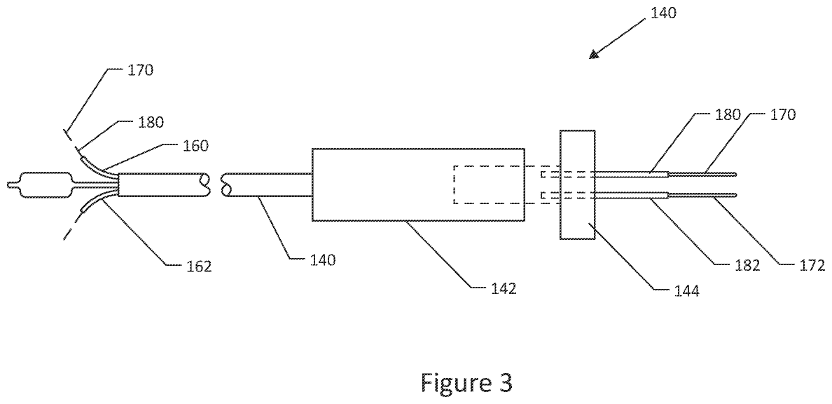

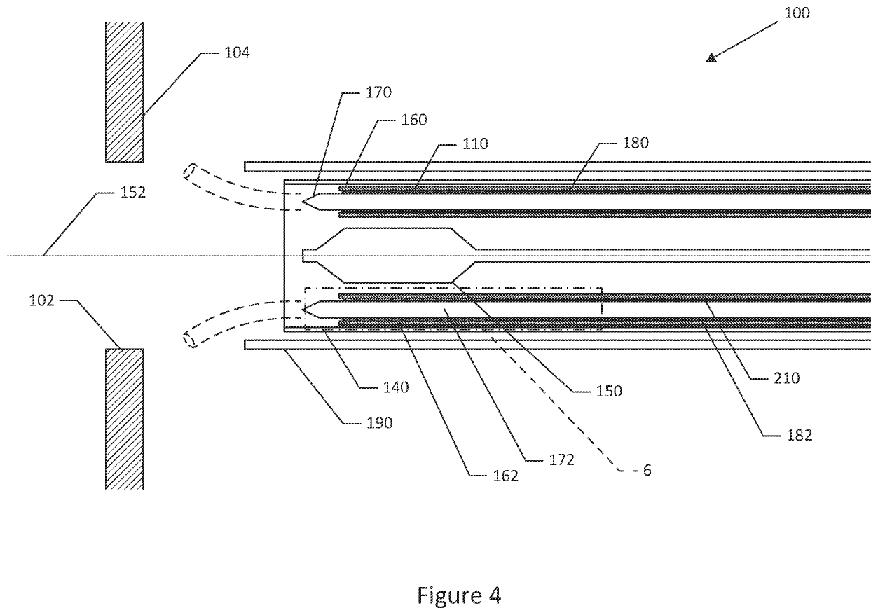

With reference to FIGS. 3 and 4, the illustrated system 100 includes a guide sheath 190 can be delivered to the heart, the atrium, and to the septal wall 104 or other desired anatomical location using imaging guidance, such as fluoroscopy and/or echocardiography, and/or using any suitable techniques known in the art. The guide sheath 190 can also permit one or more delivery catheters to be advanced to the heart, the atrium, and to the septal wall 104 or other desired anatomical location. The guide sheath 190 can be configured to receive one or more delivery catheters.

The system 100 can include a delivery catheter 140 that is configured to deliver components of the system 100, such as one or more of the tissue anchors 110, suture(s) 112, locating member 150, and suture lock 114. The system 100 can also include a handle 142. The handle 142 can receive the one or more delivery catheters.

The illustrated system 100 includes a delivery catheter 140 that can be received by the handle 142 and/or the guide sheath 190. The handle 142 can be used to manipulate the delivery catheter 140, such as to orient the distal end of the delivery catheter 140. The delivery catheter 140 can also include a driver or actuating knob 144. The actuating knob 144 can be positioned proximally to the handle 142. The actuating knob 144 can enable capabilities of the delivery catheter 140, such as deployment of components of the delivery catheter 140, such as one or more delivery tubes, one or more pushers or one or more needles, as further discussed below. The actuating knob 144 can act as a central hub to maintain positions of the delivery tubes, the pushers and the needles relative to one another. The relative positions can be maintained by friction as well as locking features (not shown). When the actuating knob 144 is actuated, the individual components (the needles, delivery tubes, and pushers) can be moved relative to one another.

The delivery catheter 140 can further include a first delivery tube 160 that is slidably supported within the delivery catheter 140. In the illustrated arrangement, the first tissue anchor 110 is disposed within a distal end of the first delivery tube 160. In this manner, the first tissue anchor 110 can be carried by the delivery catheter 140. However, the first tissue anchor 110 can be otherwise supported by, coupled to, or configured to interact with the first delivery tube 160. The illustrated system 100 also includes a second delivery tube 162 that is slidably supported within the delivery catheter 140. In the illustrated arrangement, the second tissue anchor 110 is disposed within a distal end of the second delivery tube 162. In this manner, the second tissue anchor 110 can be carried by the delivery catheter 140. However, the second tissue anchor 110 can be otherwise supported by, coupled to, or configured to interact with the second delivery tube 162. The first delivery tube 160 and the second delivery tube 162 can be positioned on opposing radial sides of the delivery catheter 140. This can maximize the distance between the first delivery tube 160 and the second delivery tube 162 within the delivery catheter 140 and thus prevent interference of the tubes 160, 162 with one another during delivery and implantation. Furthermore, the position of the tubes 160, 162 allows for the centering member 150 to be positioned between the tubes 160, 162. The exteriors of the tubes 160, 162, in combination with the interior of the delivery catheter 140, can define a path that the centering member 150 can travel. This can enable desired positioning of the centering member 150 in the desired position relative to the anchors 110. The first and second delivery tubes 160, 162 can be stainless steel.

In the illustrated arrangement, a first needle 170 is disposed within the first delivery tube 160 and a second needle 172 is disposed within the second delivery tube 162. Each of the first needle 170 and the second needle 172 is configured to be advanced from the respective one of the first delivery tube 160 and the second delivery tube 162 and to puncture the septal wall 102 or other tissue. The actuating knob 144 can be configured to advance the first delivery tube 160 and the second delivery tube 162 out of the delivery catheter 140 and the guide sheath 190. The first and second delivery tubes 160, 162 can be configured to curve when advanced out of the delivery catheter 140. The first and second delivery tubes 160, 162 can be constructed of or include a shape memory alloy, such as nitinol, which can be set to a curved or expanded configuration. The first and second delivery tubes 160 can be set to curve in a radially outward direction. The delivery tubes 160, 162 can be constrained, such as by the delivery catheter 140, until ready for deployment. The delivery tubes 160, 162 can then be advanced forward and allowed to curve such that the delivery tubes 160, 162 extend radially outward and contact locations 130, 132 adjacent and spaced from the edges of the defect 102. The delivery tubes 160, 162 can pass through the respective holes created by the first and second needles 170, 172 as described above. The delivery tubes 160, 162 can be advanced over the needles 170, 172, which are positioned through the septal wall at locations 130, 132. The first and second needles 170, 172 can also be considered wires.

With reference to FIG. 4, the first delivery tube 160 and the second delivery tube 162 can be configured to extend away from a center or longitudinal axis of the delivery catheter 140 when advanced or deployed from the delivery catheter 140. In some configurations, at least a distal end portion of each of the first delivery tube 160 and the second delivery tube 162 is constructed from or includes a shape memory alloy. In some configurations, the distal end of each of the first delivery tube 160 and the second delivery tube 162 includes a curved portion that curves radially outwardly when deployed. Accordingly, the first delivery tube 160 and the second delivery tube 162 can guide a respective one of the first needle 170 and the second needle 172 along a curved path to reach the respective locations 130, 132, as represented by the dashed line position of FIG. 4. As illustrated, the first delivery tube 160 and the second delivery tube 162, and therefore the first needle 170 and the second needle 172, curve in an opposing directions from each other.

Other arrangements for directing the first delivery tube 160 and the second delivery tube 162 and/or the first needle 170 and the second needle 172 away from the center or longitudinal axis of the delivery catheter 140 can also be used. For example, the first delivery tube 160 and the second delivery tube 162 may not be pre-shaped but can be flexible so that they can be deflected away from the center or longitudinal axis. In some configurations, a curved or ramped surface may be provided to deflect the first delivery tube 160 and the second delivery tube 162 to a desirable angle relative to the center or longitudinal axis. In some configurations, the deflection of the first delivery tube 160 and the second delivery tube 162 and/or the first needle 170 and the second needle 172 can be configured to allow the tissue anchors 110 to be located at the first location 130 and the second location 132 that preferably is between about 3 mm to 5 mm from the respective first edge and the second edge of the atrial septal defect or other tissue opening 102, as described above.

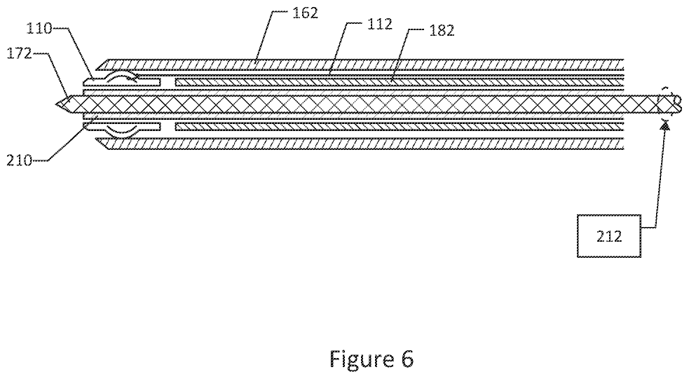

In some configurations, each of the first needle 170 and the second needle 172 are conductive or include a conductive material. With such an arrangement, radiofrequency (RF) energy can be applied to the first needle 170 and the second needle 172 to facilitate puncturing of the septal wall 104 or other tissue. With reference to FIG. 6, a proximal end or all but the distal end portions of the first needle 170 and the second needle 172 can include an insulation covering or an outer insulating member 210 such that the RF energy is directed to the distal end portions of the first needle 170 and the second needle 172 and does not get transferred to other components of the system 100. Any suitable RF generator 212 can be used, such as those sold by the Bovie Medical Corporation of Clearwater, Florida. A grounding pad can be applied to the patient and connected to the RF generator 212, as is known in the art.

As shown in FIG. 6, the tube 162, the anchor 110 and the needle 172 can together form a chamfered profile. The distal end of the needle 172 can extend the furthest distally out of the tube 162. The distal end of the anchor 110 can be positioned proximally from the distal end of the needle 172, such that the distal end of the anchor 110 extends distally less than the distal end of the needle 172. The distal end of the tube 162 can be positioned proximally from the distal end of the anchor 110, such that the distal end of the tube 162 can distally extend less than the distal end of the anchor 110. This chamfered profile can advantageously allow the needle 172, anchor 110, and tube 162 to cooperate to puncture through the septal wall. Once in position through the septal wall, the anchor 110 can be deployed from the distal end of the tube 162.

The illustrated system 100 includes a first pusher 180 and a second pusher 182. The first pusher 180 and the second pusher 182 are slidably received within a respective one of the first delivery tube 160 and the second delivery tube 162. The first pusher 180 is located behind the first tissue anchor 110 relative to the distal tip of the delivery catheter 140. Similarly, the second pusher 182 is located behind the second tissue anchor 110 relative to the distal tip of the delivery catheter 140. Each of the first pusher 180 and the second pusher 182 can be or comprise a tube that surrounds a respective one of the first needle 170 and the second needle 172. Each of the first pusher 180 and the second pusher 182 can be hollow.

Each of the first pusher 180 and the second pusher 182 is configured to move the respective one of the first tissue anchor 110 and the second tissue anchor 110 relative to the respective one of the first delivery tube 160 and the second delivery tube 162. The first pusher 180 and the second pusher 182 can be moved in a distal direction, which in turn can push or move the respective one of the first tissue anchor 110 and the second tissue anchor 110 in a distal direction. The first tissue anchor 110 or the second tissue anchor 110 can be pushed out the distal end of the respective first or second delivery tube 160, 162. Accordingly, the first pusher 180 and the second pusher 182 can implant, allow for implantation, or otherwise facilitate implantation of the respective one of the first tissue anchor 110 and the second tissue anchor 110 into the septal wall 104 or other tissue. The first and second tissue anchors 110 can be implanted simultaneously, sequentially, or temporally overlapping.

As illustrated in FIG. 1, the first tissue anchor 110 and the second tissue anchor 110 can be connected to one another by one or more sutures 112. The suture(s) 112 can be used to draw together the implanted tissue anchors 110 to reduce the size of or close the tissue opening 102. A suture lock 114 can be employed to fix an effective length of the suture(s) 112 between the tissue anchors 110. The suture(s) 112 can be secured to the tissue anchors 110 prior to implantation of the tissue anchors 110. For example, the suture(s) 112 can be coupled to the tissue anchors 110 prior to use of the system 100. As illustrated in FIG. 3, an end of the suture(s) 112 can be accessible from a proximal end of the delivery catheter 140 or other proximal portion of the system 100 to allow manipulation of the suture(s) 112.

In some embodiments, the suture lock 114 is applied to the suture(s) 112 after implantation of the tissue anchors 110 and is advanced to the implanted tissue anchors 110 through the delivery catheter 140 or a separate delivery catheter that is introduced through the guide sheath 190.

The illustrated system 100 includes a locating member 150 that is configured to locate the delivery catheter 140 relative to the tissue opening. The locating member 150 can be configured to center or approximately center the delivery catheter 140 relative to the tissue opening 102. Accordingly, the locating member 150 can be referred to as a centering member. The centering member 150 can be or comprise a tube or a catheter. Accordingly, the centering member 150 can be referred to as a centering tube or centering catheter. The centering member 150 is slidably supported within the delivery catheter 140. The centering member 150 is configured to extend from the delivery catheter 140 into the tissue opening 102. The centering member 150 can be coaxially received by the delivery catheter 140. The centering member 150 and the delivery catheter 140 can be coaxially arranged onto or relative to a guide wire 152, which can be advanced through vasculature to the atrium. The guide wire 152 can be used to guide and deliver the centering member 150 and the delivery catheter 140 to the atrium. As describer further herein, the centering member 150 can be configured to complement or generally or substantially conform to the shape of the elongated tissue opening 102. In some configurations, the centering member 150 defines a non-circular outside shape. In some configurations the centering member defines a circular outside shape.

As described above, the locating member or centering member 150 preferably is configured to locate the delivery catheter 140 relative to the tissue opening. A rotational position of the centering member 150 relative to the delivery catheter 140 can be fixed or at least determinable such that a location of the first delivery tube 160 and the second delivery tube 162 and, thus, the first and second tissue anchors 110 relative to the centering member 150 can be established. The centering member 150 can be configured to center or approximately center the delivery catheter 140 relative to the tissue opening 102 and to position the first delivery tube 160 and the second delivery tube 162 at a respective one of the first location 130 and the second location 132.

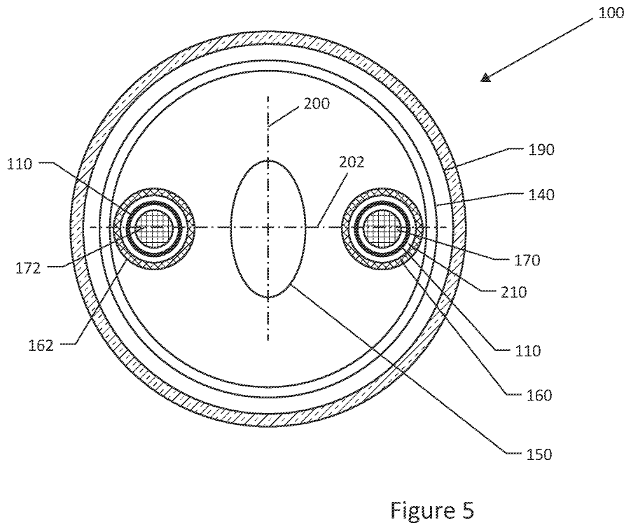

With reference to FIGS. 4 and 5, the centering member 150 defines a non-circular outside shape when viewed from the distal end or along a longitudinal axis of the delivery catheter 140 and/or centering member 150. In the illustrated arrangement, the non-circular outside shape is an oval or elliptical shape. The centering member 150 can be a substantially ellipsoid balloon. As described above, the outside shape can also be circular. In the illustrated arrangement, the non-circular outside shape of the locating member defines a major axis 200 and a minor axis 202. The centering member 150 is configured such that the major axis 200 and the minor axis 202 can be generally aligned or at least substantially aligned with the major axis 120 and the minor axis 122, respectively, of the tissue opening 102 (FIG. 2). As a result, the first delivery tube 160 and the second delivery tube 162 can be properly positioned such that the first tissue anchor 110 and the second tissue anchor 110 are located for implantation generally at or at least substantially at the first location 130 and the second location 132, respectively.

The illustrated arrangement can reduce or eliminate the dependency on guidance techniques to properly locate the tissue anchors 110 once the centering member 150 is engaged with the tissue opening 102. Accordingly, placement of the tissue anchors 110 can be easier, quicker, and/or more accurate than reliance on guidance alone to position the tissue anchors 110 relative to the tissue opening 102. This is especially true of the rotational alignment of the tissue anchors 110 relative to the tissue opening 102. It can be desirable to position the tissue anchors 110 relatively close to the edge of the tissue opening 102 while leaving enough tissue between the tissue anchor 110 and the edge of the tissue opening 102 to inhibit or prevent tearing of the tissue.

The centering members 150 described herein can facilitate accurate positioning of the tissue anchors 110 relative to the edges of the tissue opening 102 in view of the limitations inherent in guidance and imaging techniques. In some configurations, system 100 is configured such that the first location 130 is between about 3 mm to 5 mm from a first edge of the atrial septal defect or other tissue opening 102, and the second location 132 is between about 3 mm to 5 mm from a second edge of the atrial septal defect or other tissue opening 102. In some configurations, the first tissue anchor 110 and the second tissue anchor 110 are the only tissue anchors of the system 100. When properly positioned, only two tissue anchors 110 can be sufficient to reduce the size of or close the tissue opening 102. However, in other arrangements, additional tissue anchors 110 could be deployed, and could be arranged in stacked pairs of tissue anchors 110 along the major axis 120 of the tissue opening 102, for example.

As illustrated in FIG. 5, the first delivery tube 160 and the second delivery tube 162, and therefore the first and second tissue anchors 110, are equally spaced on opposite sides of the major axis 200. In the illustrated arrangement, the first and the second tissue anchors 110 are located on the minor axis 202.

The centering member 150 can be of any suitable arrangement to properly locate and orient the delivery catheter 140 or other component of the system 100 relative to the tissue opening 102 as described above. In some configurations, as illustrated in FIG. 4, the centering member 150 is or includes a non-circular balloon. The balloon 150 can have a non-circular shape only in an expanded or deployed configuration or can have a non-circular shape in both the non-deployed and the deployed configurations. The centering member 150 can also be a circular balloon.

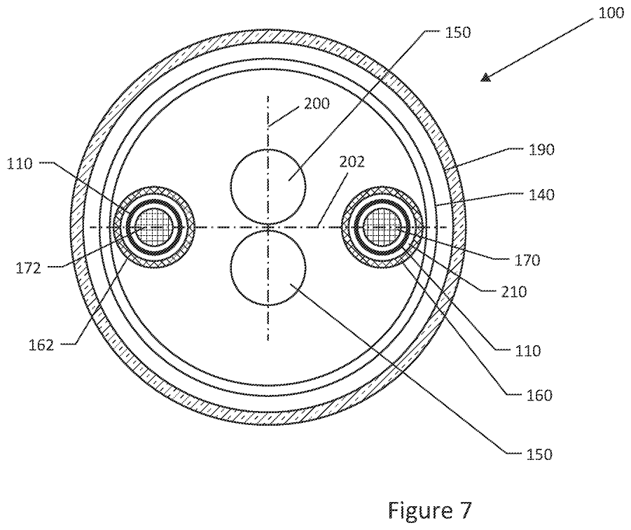

In some configurations, the centering member 150 can be or include at least two balloons. As illustrated in FIG. 7, the centering member 150 includes two balloons in a stacked configuration along the major axis 200. One or more of the at least two balloons can be circular. In the illustrated arrangement, both balloons are circular. However, non-circular shapes could also be used. In some examples, the at least two balloons can together form a circular or a non-circular shape.

The balloons can be expandable to a deployed condition by techniques as known in the art. In some configurations, the balloon(s) are semi-compliant or compliant to allow the balloons to conform to the shape of the opening 102.

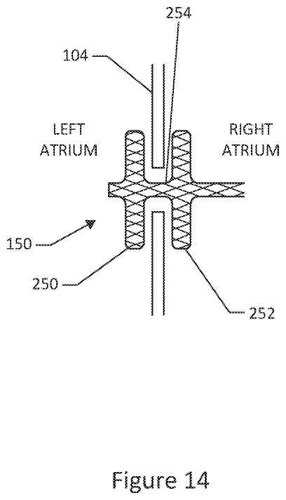

In some configurations, as shown in FIG. 14, the centering member 150 is or includes an expandable body, such as a cage or trusswork. In some configurations, the expandable body comprises a shape memory alloy. In some configurations, the expandable body is self-expanding once deployed from the delivery catheter 140.

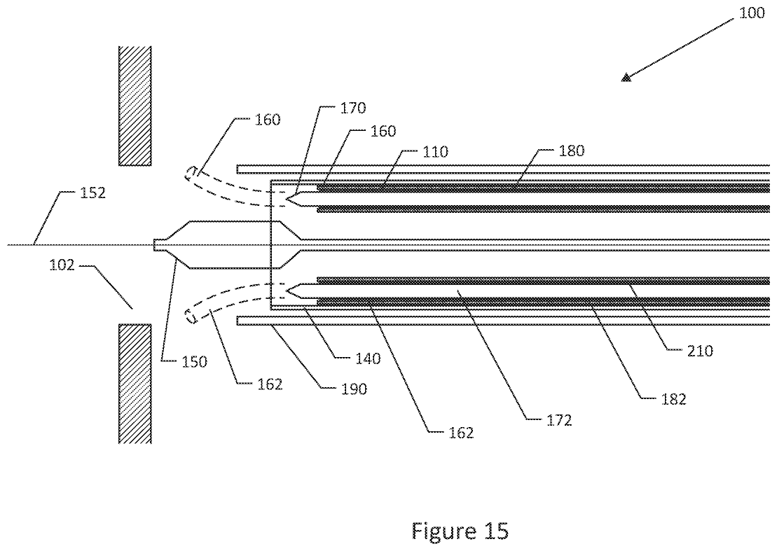

FIG. 15 is a sectional view of a distal end portion of a portion of an alternative version of the system in which the centering balloon is external of a catheter of the system. The system 100 of FIG. 15 differs from the systems 100 shown in FIGS. 3-5 or FIG. 7 in that the centering balloon 150 is positioned outside of the delivery catheter 140 and/or axially beyond or distally of the first delivery tube 160 and the second delivery tube 162. Such an arrangement provides additional space for the centering balloon 150 in the collapsed introduction configuration. For example, the radial dimensions of the centering balloon 150 are not constrained by the presence of the first and second delivery tubes 160, 162. The radial dimensions of the centering balloon 150 in the collapsed introduction configuration can be as large as the delivery catheter 140 or as large as can be passed through the guide sheath 190. In other respects, the system 100 of FIG. 15 can be the same as or substantially similar to the other systems 100 disclosed herein, or can be of another suitable arrangement. Accordingly, features or structures not discussed in detail can be assumed to be the same as or similar to the corresponding features or structures of the other systems 100 disclosed herein, or can be of another suitable arrangement.

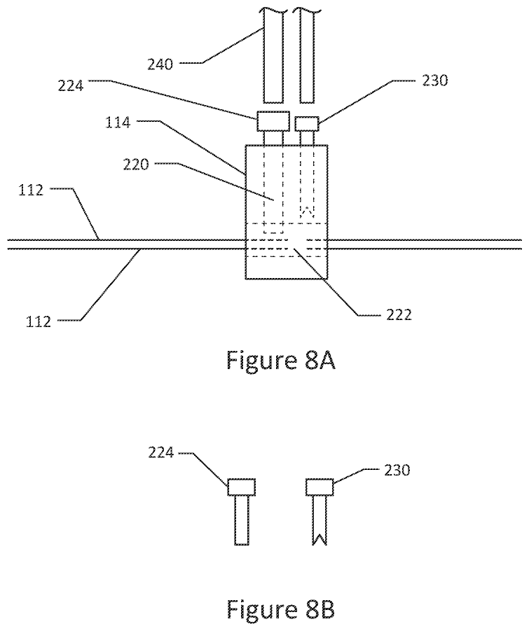

As described above, a suture lock 114 can be used to fix the effective length of a tensioned portion of the suture(s) 112 between the tissue anchors 110. Any suitable type of suture lock 114 can be used, including a knot in the suture(s) 112, for example. However, FIG. 8 includes a possible suture lock 114.

FIG. 8A illustrates a suture lock 114 that also serves as a suture cutter. The illustrated suture lock 114 includes a body 220, which has a passage 222 through which the suture 112 is passed. The illustrated body 220 is generally rectangular in shape and the passage 222 passes in a radial direction through the body 220. The rectangular shape of the body 220 can advantageously provide rotational control of the suture lock 114, which can prevent the suture lock 114 from undesirable spinning. In other embodiments, the body 220 can be generally cylindrical. A lock element, such as a lock screw 224 as shown in FIG. 8B, can be supported by the body 220 and can be configured to be advanced into the passage 222 to press and capture a portion of the suture 112 against an opposite surface of the passage 222 to secure the body 220 at a desired location along the suture 112. The body 220 can be positioned against one of the tissue anchors 110 if a single suture 112 is used to fix the effective tensioned length of the suture 112 between the tissue anchors 110. If multiple sutures 112 are used, a portion of each suture 112 can be passed through the passage 222 and the body 220 can be located between the tissue anchors 110 and can similarly fix a tensioned length of the sutures 112 between the tissue anchors 110.

The suture lock 114 of FIG. 8 also includes a cutting element, such as a cutting screw 230 as shown in FIG. 8B. The cutting screw 230 can be supported by the body 220 and can have a sharp end. The cutting screw 230 can be advanced into the passage 222 to cut the suture 112. In some configurations, one or both of the lock screw 224 and the cutting screw 230 are moved relative to the body 220 by one or more drivers 240. In some configurations, each of the lock screw 224 and the cutting screw 230 has a dedicated driver 240.

In use, an exposed portion of the suture(s) 112 can be passed through the passage 222 of the body 220 after implantation of the tissue anchors 110. The body 220 can be advanced through the delivery catheter 140 or another delivery catheter to the location of the tissue anchors 110. The driver(s) 240 can be engaged with the lock screw 224 and the cutting screw 230 prior to the body 220 being passed through the delivery catheter 140. In some configurations, the drivers 240 can be used to advance or to assist with the advancement of the body 220 through the delivery catheter 140.

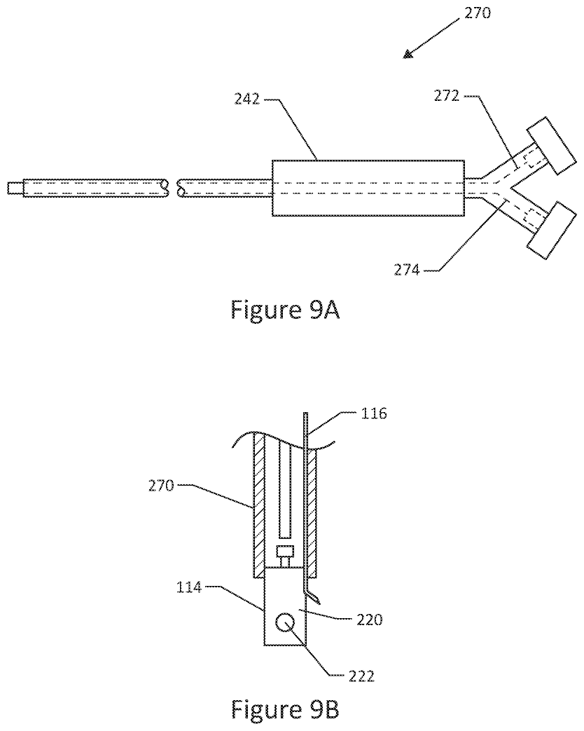

As described above, the delivery catheter 140 can be used to deliver the suture lock 114. In other embodiments, the system 100 can include a second delivery catheter 270, as shown in FIG. 9A. The second delivery catheter 270 can be a suture lock delivery catheter. Similar to the first delivery catheter 140, the second delivery catheter 270 can be received by a handle 242 and/or the guide sheath 190. The handle 242 can be used to manipulate the second delivery catheter 270, such as to orient the distal end of the second delivery catheter 270.

The second delivery catheter 270 can include one or more drive shafts. As shown in FIG. 9A, the second delivery catheter 270 can include two drive shafts, a first drive shaft 272 and a second drive shaft 274. Each of the drive shafts 272, 274 can include a rotating knob to actuate each of the drive shafts 272, 274. The drive shafts 272, 274 can enable capabilities of the second delivery catheter 270, such as activation of components of the second delivery catheter 270, such as the suture lock and cutter 114. For example, the first drive shaft 272 can move a first driver 240, which can in turn move the locking screw 224. The locking screw 224, when moved by the first driver 240, can be advanced within the passage 222 and lock the suture 112. Similarly, the second drive shaft 274 can move a second driver 240, which can in turn move the cutting screw 230. The cutting screw 230, when moved by the driver 240, can advance into the passage 222 and cut the suture 112.

The body 220 of the suture lock 114 can be positioned at a distal end of the second delivery catheter 270 as shown in FIG. 9B. The second delivery catheter 270 can include a retaining wire 116 positioned between the body 220 of the suture lock 114. The retaining wire 116 can be wedged between the body 220 of the suture lock 114 and an interior wall of the delivery catheter 270. The retaining wire 116 being positioned between the body 220 of the suture lock 114 can create an interference fit that retains the suture lock 114 within the suture lock delivery catheter 270 until it is released. When the retaining wire 116 is removed, such as by retraction, the suture lock 114 can be released and be deployed from a distal end of the second delivery catheter 270.

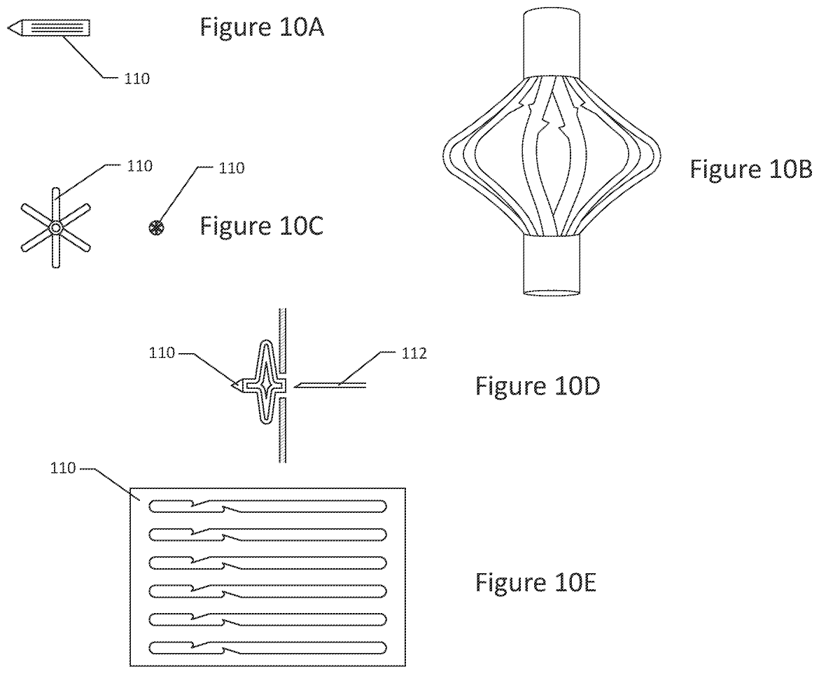

Any suitable type of tissue anchor 110 can be employed, including barb-type tissue anchors. FIG. 10A illustrates one embodiment of a tissue anchor 110 in the form of a tube having one or more slots extending in a longitudinal direction. The tube 110 can be constructed of or include a shape memory alloy, such as nitinol, and can be set to an expanded configuration in which ends of the tube are brought towards one another such that the side wall of the tube 110 flares in a radially outward direction, as shown in FIG. 10B. In this manner, the tissue anchor 110 can be self-expanding. This allows the tissue anchor 110 to transform from a first configuration to a second configuration. In the first or introduction configuration, the tissue anchor 110 can be radially collapsed and elongate, such that the tissue anchor 110 can fit and be constrained within a delivery tube. The tissue anchor 110 can be constrained, such as by the delivery tubes 160, 162, until passed through the septal wall or other tissue wall. In the second or deployed configuration, the tissue anchor 110 can radially expanded and shorter in length than in the first configuration. FIGS. 10B and 10C illustrate the second configuration in which the tissue anchor 110 is radially expanded. The tissue anchor 110 can be advanced out of the delivery tube and then allowed to expand such that the tissue anchor 110 can no longer pass through the opening in the septal wall or other tissue wall. This can result in the tissue anchor 110 being anchored in place positioned within an opening of the septal wall and against the septal wall, as shown in FIG. 10D. FIG. 10E illustrates the laser cut pattern of the arms of the tissue anchor 110. As illustrated, the arms can include notches and/or barbs, which can further allow the tissue anchor 110 to anchor in place against a tissue wall, such as by a barb piercing the tissue wall. The tissue anchor 110 can have any number of arms, such as 6 arms as shown in FIGS. 10B and 10C. In other examples, the tissue anchor 110 can have another number of arms, such as 2 arms, 3 arms, 4 arms, 5 arms, 7 arms, 8 arms, 9 arms, or 10 arms.

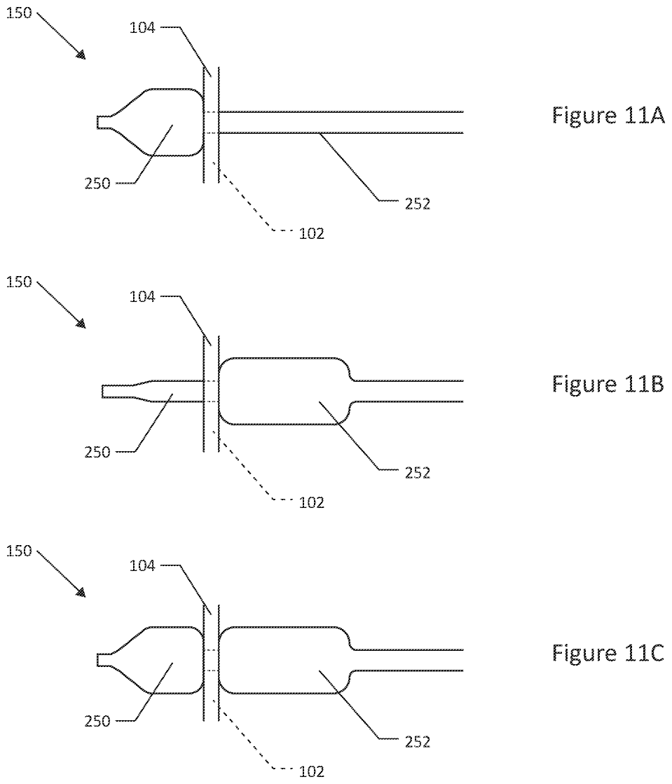

FIGS. 11A, 11B, and 11C illustrate several example centering balloons 150 within a septal defect or other tissue opening 102. At least some of the illustrated centering balloons 150 can vary in size along a length of the centering balloon 150. In some embodiments, a radial dimension of the centering balloon 150 varies along its length or longitudinal axis. In some embodiments, at least a vertical dimension of the centering balloon 150 varies along its length or longitudinal axis. In some embodiments a horizontal dimension of the centering balloon 150 varies along its length instead of or in addition to the vertical dimension. The centering balloon 150 can have an indicator (e.g., a radiopaque marker) along its length to facilitate positioning of the centering balloon 150 relative to the septal wall 104. In some embodiments, the indicator can be located at or near a midpoint of the centering balloon 150. As described herein, the centering balloon 150 can be configured to center or approximately center the delivery catheter 140 relative to a tissue opening 102. The centering balloon 150 is configured to extend into the tissue opening 102. The centering balloon 150 can have a first configuration in which the balloon is deflated and all portions of the centering balloon are radially collapsed. The deflated centering balloon 150 can be folded and/or compressed to fit within the delivery catheter 140. The centering balloon 150 can then be placed in position, such that the distal portion 250 is placed on a first side of the septal wall and the proximal portion 252 is placed on a second side of the septal wall. The centering balloon can then be inflated such that the distal portion 250 and/or the proximal portion 252 can each expand in a radial dimension such that the centering balloon is axially constrained. In this manner, the centering balloon 150 can act as a temporary anchor to position the delivery catheter 140 in place. The centering balloon 150 can be deflated and removed from the septal opening when desired, such as after implantation of the tissue anchors 110. The centering balloon 150 can be molded into a desired shape. The centering balloon 150 can additionally or alternatively have a varying thickness to achieve the varying radial dimension along the length of the centering balloon 150. For example, the portions of the centering balloon with a larger radius in the expanded configuration can have a thinner wall thickness than the thickness of the wall of the portions of the centering balloon with a smaller radius.

In some embodiments, the centering balloon 150 can vary in size on opposing sides of the indicator. FIG. 11A illustrates a centering balloon 150 having a distal portion 250 that is larger in a radial dimension in comparison to a proximal portion 252 of the centering balloon 150. The centering balloon 150 can be positioned such that the larger distal portion 250 is located on one side (e.g., a distal side) of the septal wall 104 and the smaller proximal portion 252 can be located on the other side (e.g., a proximal side) of the septal wall 104. Such an arrangement can provide greater resistance to axial movement of the centering balloon 150 in a proximal direction than in a distal direction; however, resistance in the distal direction can still be provided.

FIG. 11B illustrates a centering balloon 150 having a proximal portion 252 that is larger in a radial dimension in comparison to a distal portion 250. The centering balloon 150 can be positioned such that the larger proximal portion 252 is located on the proximal side of the septal wall 104 and the smaller distal portion 250 can be located on the distal side of the septal wall 104. Such an arrangement can provide greater resistance to axial movement of the centering balloon 150 in a distal direction than in a proximal direction; however, resistance in the proximal direction can still be provided.

FIG. 11C illustrates a centering balloon 150 in which a distal portion 250 and a proximal portion 252 have the same or similar radial dimension, such as a vertical and/or a horizontal dimension. Such an arrangement can provide equal or substantially equal resistance to axial movement of the centering balloon 150 in both the proximal and distal directions.

In other examples, instead of a distal portion 250 and a proximal portion 252, the centering member can instead include two balloons, a distal balloon and a proximal balloon. This dual balloon centering member can operate similarly to the centering balloon 150 described above. Rather than the intermediate portion being a balloon portion connecting the distal portion and the proximal portion, the intermediate portion can be a suture, a wire, or any other means to connect a distal balloon and a proximal balloon.

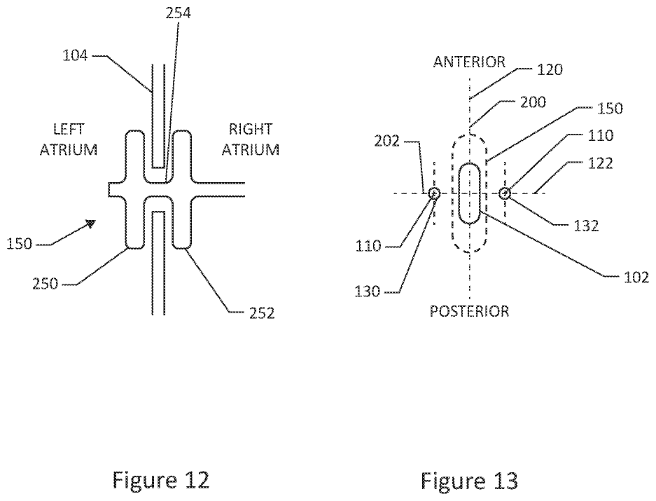

FIGS. 12 and 13 illustrate another version of the centering balloon 150 in which each of the distal portion 250 and the proximal portion 252 have the same or similar radial dimension, such as a vertical and/or a horizontal dimension. An intermediate portion 254 between the distal portion 250 and the proximal portion 252 can have a radial dimension, such as a vertical and/or a horizontal dimension, that is smaller than the corresponding radial dimension of one or both of the distal portion 250 and the proximal portion 252. The distal portion 250 and the proximal portion 252 have a smaller axial dimension in comparison to an axial dimension of one or both of the distal portion 250 and the proximal portion 252 of the centering balloon 150 of FIG. 10c. An axial dimension of the distal portion 250 and the proximal portion 252 can be approximately equal to a spacing between them or an axial dimension of the intermediate portion 254. The axial dimensions may vary with inflation of the centering balloon 150.

FIG. 13 shows an end view of the centering balloon 150 size and position relative to the septal wall defect 102 and the tissue anchor locations 130, 132. The tissue anchors 110 are also shown at the respective tissue anchor locations 130, 132. As illustrated, at least the distal portion 250 and the proximal portion 252 have a radial dimension that is larger than a corresponding dimension of the septal wall defect 102. In the illustrated arrangement, both the vertical and the horizontal radial dimensions (or dimensions along the major axis 200 and the minor axis 202) are larger than the corresponding dimensions of the septal wall defect 102. The vertical radial dimension may be at least 2-3 times the horizontal radial dimension. The horizontal radial dimension is selected to be smaller than the horizontal dimension between the tissue anchor locations 130, 132 to allow for placement of the tissue anchors 110 with the centering balloon 150 inflated or expanded and the tissue anchors 110 are implanted at tissue anchor locations 130, 132 as disclosed herein.

FIG. 14 illustrates another version of the centering member 300. The centering member 300 can include an expandable cage. The expandable cage centering member 300 can define a noncircular shape, similar to the centering balloon 150 described above. In some examples, the expandable cage centering member 300 can define a circular shape. The expandable cage centering member 300 can include an elongate shape that can vary in size (i.e. radial dimension) along a length of the expandable cage centering member 300. In some examples, the expandable cage centering member 300 can have a similar profile or shape as the centering balloon 150 as shown in FIG. 12. The centering member 300 can include an intermediate portion 354 between a distal portion 350 and a proximal portion 352 that is smaller in radial dimension than the corresponding radial dimension of one or both of the distal portion 350 and the proximal portion 352. In the same manner as the centering balloon 150 as described above, the centering member 300 can transform from a radially collapsed configuration to a radially expanded configuration. The centering member 300 can be delivered and placed through an opening in a tissue wall, such as a septal wall, in the collapsed configuration. Once in the desired location, the centering member 300 can be radially expanded. The centering member 300 can act as a temporary anchor, such as for a delivery catheter. The expandable cage centering member 300 can be a self-expanding anchor, such that it is constrained by a catheter and expand when advanced out of the catheter. The expandable cage centering member 300 can also be manipulated between the expanded and collapsed positions, such as by a driveshaft.

As described herein, the delivery catheter 140 is configured to deliver components of the system 100, such as the tissue anchors 110, suture(s) 112, and suture lock 114. The centering balloon 150 is configured to locate the delivery catheter 140 relative to the tissue opening. The centering balloon 150 can be configured to center or approximately center the delivery catheter 140 relative to the tissue opening 102. The centering balloon 150 is configured to extend into the tissue opening 102. The centering balloon 150 can be coaxially received by the delivery catheter 140 and both can be coaxially arranged onto or relative to the guidewire 152, which can be advanced through vasculature to the atrium. The centering balloon 150 can define a non-circular outside shape by itself or in combination with one or more other balloons 150, as described herein. In some examples, the centering balloon 150 can define a circular shape by itself or in combination with one or more other balloons.

The illustrated system 100 includes the first delivery tube 160 and the second delivery tube 162 that are slidably supported within the delivery catheter 140. The first tissue anchor 110 is disposed within a distal end of the first delivery tube 160 and the second tissue anchor 110 is disposed within a distal end of the second delivery tube 162. However, the second tissue anchor 110 can be otherwise supported by, coupled to, or configured to interact with the second delivery tube 162. The first needle 170 is disposed within the first delivery tube 160 and the second needle 172 is disposed within the second delivery tube 162. Each of the first needle 170 and the second needle 172 is configured to be advanced from the respective one of the first delivery tube 160 and the second delivery tube 162 and to puncture the septal wall 102 or other tissue.

The system 100 also includes the first pusher 180 and the second pusher 182 that are slidably received within a respective one of the first delivery tube 160 and the second delivery tube 162. The first pusher 180 and the second pusher 182 are located behind a respective one of the first tissue anchor 110 and the second tissue anchor 110 relative to the distal tip of the delivery catheter 140. Each of the first pusher 180 and the second pusher 182 is configured to move the respective one of the first tissue anchor 110 and the second tissue anchor 110 relative to the respective one of the first delivery tube 160 and the second delivery tube 162. Accordingly, the first pusher 180 and the second pusher 182 can implant, allow for implantation, or otherwise facilitate implantation of the respective one of the first tissue anchor 110 and the second tissue anchor 110 into the septal wall 104 or other tissue. The first and second tissue anchors 110 can be implanted simultaneously, sequentially, or temporally overlapping.

The first tissue anchor 110 and the second tissue anchor 110 can be connected to one another by one or more sutures 112 (FIG. 1). The suture(s) 112 can be used to draw together the implanted tissue anchors 110 to reduce the size of or close the tissue opening 102. A suture lock 114 (FIG. 1) can be employed to fix an effective length of the suture(s) 112 between the tissue anchors 110, as described herein.

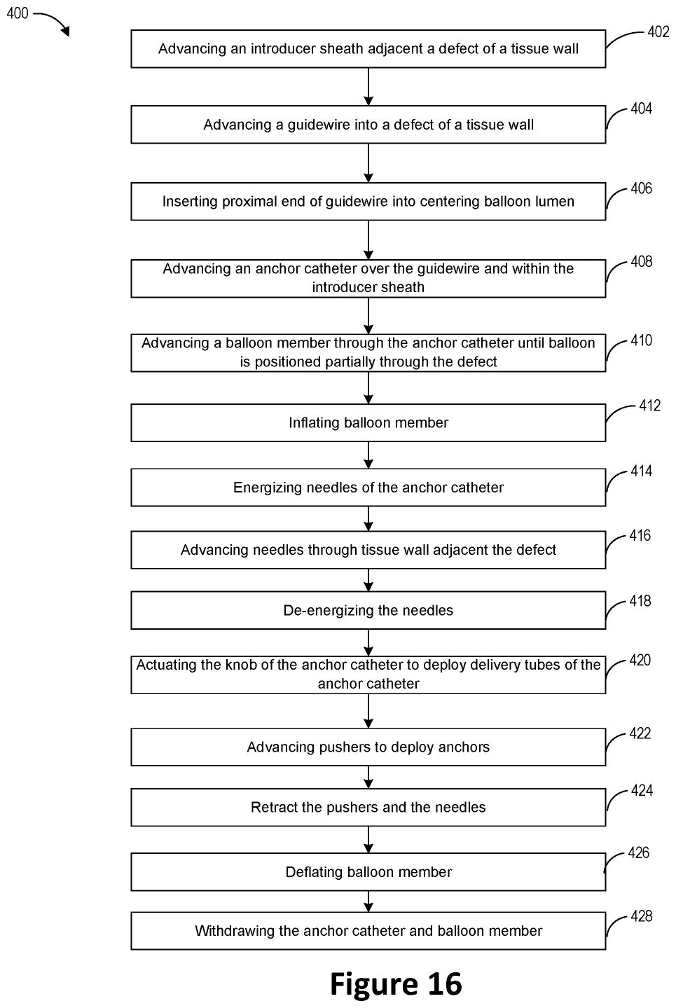

FIG. 16 is a flow diagram, illustrating a method 400 of repairing a tissue defect. Although described in context of an atrial septal defect, the method can be used to repair any tissue defect. In some examples, the tissue defect can be an atrial septal defect, such as an iatrogenic atrial septal defect or iASD.

Initially, at block 402, an introducer sheath or guide sheath 190 can be positioned adjacent a septal defect 102. In some examples, an introducer sheath or guide sheath 190 can be introduced via the femoral vein into the heart and positioned adjacent the septal defect 102. An example of an introducer sheath 190 is shown in FIGS. 4 and 5. In some examples, the introducer sheath can be positioned against a first side of the septal wall, which can be the right atrium side of the septal wall. In some examples, the introducer sheath or guide sheath 190 can be introduced through the femoral vein into the heart and positioned adjacent the septal defect 102 by advancing the introducer sheath 190 over a guidewire.

At block 404, a guidewire can be positioned through the introducer sheath 190 such that the distal end of the guidewire 152 is passed through the septal defect 102. In some examples, the guidewire 152 can be introduced prior to the introducer sheath 190 being introduced. The guidewire 152 can be positioned through the septal defect 102. In some examples, the guidewire 152 can be advanced through the introducer sheath 190 and the distal end of the guidewire 152 can be deployed from the distal end of the introducer sheath 190. The guidewire 152 can then be passed through the tissue defect. An example of a guidewire 152 is shown in FIG. 4. In some examples, the guidewire 152 can be 0.035 inches in diameter. In other examples, the guidewire 152 can be between 0.014 to 0.038 inches in diameter.

At block 406, the proximal end of the guidewire 152 can be inserted into the lumen of the centering balloon or member 150. An example of the centering balloon 150 can be seen in FIGS. 4 and 5.

At block 408, an anchor catheter 140 can be advanced within the introducer sheath 190 over the guidewire 152. The distal end of the anchor catheter 140 can be positioned adjacent to the tissue defect. The distal end of anchor catheter 140 can be positioned in contact with the tissue wall. In some examples, a peel-away sheath (not shown) can optionally be used to facilitate entry of the anchor catheter 140 into the introducer sheath 190. The peel-away sheath can then be peeled away and removed after the anchor catheter 140 is placed in the introducer sheath 190. An example of the anchor catheter 140 is shown in FIGS. 3-5. As previously described, the anchor catheter 140 can slidably receive one or more of a centering member 150, at least two delivery tubes 160, 162, at least two needles 170, 172, at least two pushers 180, 182, and at least two anchors 110. As shown in FIGS. 4 and 5, the anchor catheter 140 can receive a first delivery tube 160, a second delivery tube 162, and a centering member 150. The first delivery tube 160 can receive an anchor 110, a suture 112 attached to the anchor 110, a first needle 170 passing through the central lumen of the anchor 110, and a pusher 180 positioned behind the anchor 110. As shown in FIG. 6, the second delivery tube 162 can similarly receive a second anchor 110, a suture 112 attached to the second anchor 110, a second needle 172 passing through a central lumen of the second anchor 110, and a second pusher 182 positioned behind the second anchor 110. Each of the first and second needles 170, 172 can have insulation at the distal end of each needle. An RF generator 212 can be attached to the uninsulated proximal end of each needle. While the anchor catheter 140 is advanced over the guidewire 152, the components therein are also advanced over the guidewire 152, including the centering member 150.

At block 410, the balloon member 150 can be advanced relative to the anchor catheter 140. The distal end of the balloon member 150 can be deployed out of the distal end of the anchor catheter 140 until the balloon member 150 is positioned partially through the defect. As described previously, the balloon member 150 can include an indicator (such as a radiopaque marker) to facilitate positioning of the balloon member 150 relative to the septal wall.

At block 412, the balloon member 150 can be inflated. The inflation process can be similar to the process described in FIGS. 11A-11C. The septal wall 104 can be held by the proximal section 252 and/or the distal section 250 of the balloon member 150 to precisely locate the anchor catheter 140.

At block 414, the first and second needles 170 and 172 can be energized. As described herein, radiofrequency (RF) energy can be applied to the first needle 170 and the second needle 172.

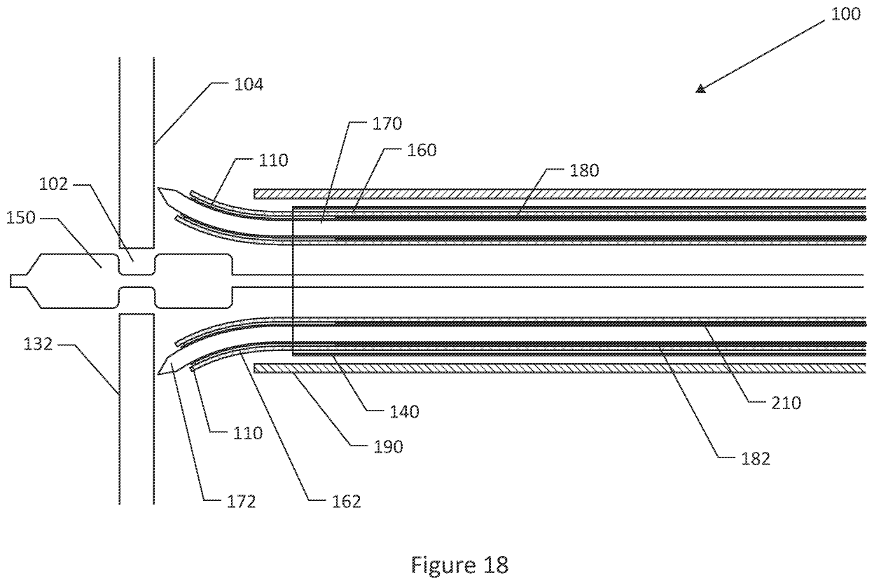

At block 416, the first and second needles 170, 172 are advanced through the septal wall 104. The needles 170, 172 being energized, as described at block 414, can facilitate puncturing of the septal wall 104. As illustrated in FIG. 2, the needles 170, 172 can puncture the septal wall at a first location 130 and a second location 132 near the edge of the tissue opening 102 and long or near the minor axis of the tissue opening 102. The first and second needles 170, 172 can be advanced through the septal walls 104 at locations 130, 132. As described herein, the locations 130, 132 can be positioned between 3 mm to 5 mm from the respective first edge and second edge of the defect 102. As shown in FIG. 18, the delivery tubes 160, 162 can be advanced out of the distal end of the anchor catheter 140 along with the corresponding needles 170, 172 and anchors 110. When advanced such that the distal ends are no longer constrained by the delivery catheter 140, the distal ends of the delivery tubes 160, 162 can curve radially outward, as illustrated in FIGS. 4 and 18 and as described herein. The needles 170, 172 and anchors 110 can be positioned at the distal ends of the delivery tubes 160, 162, as illustrated in FIGS. 6 and 18. As the needles 170, 172 are positioned within the curved portion of the delivery tubes 160, 162, the needles 170, 172 can be positioned adjacent to the locations 130, 132. This allows the needles 170, 172 to be advanced through the septal wall 102 at the locations 130, 132.

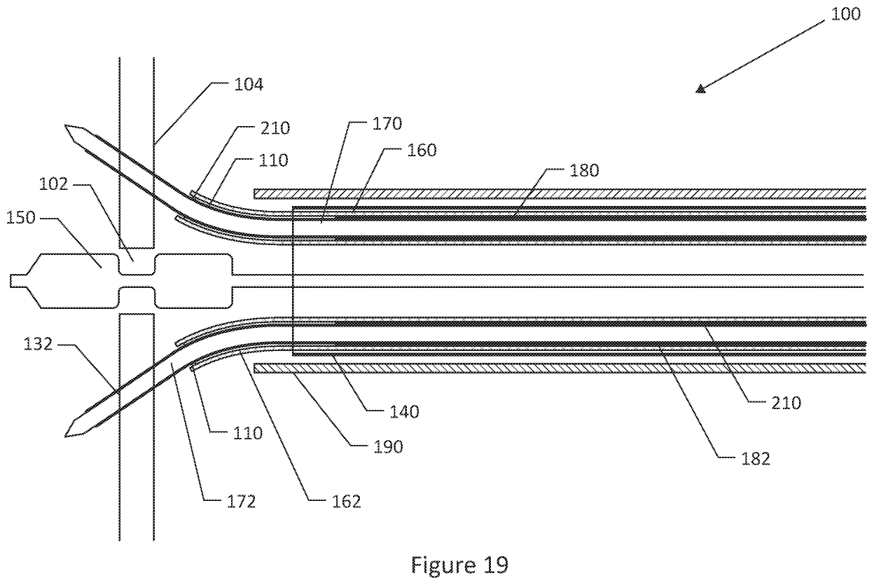

As illustrated in FIG. 19, the needles 170, 172 can be pushed through the septal wall such that they each extend beyond the septal wall by at least the length of the tissue anchor. This allows the needles 170, 172 to act as a guidewire for each of the tissue anchors to facilitate advancement and deployment of the tissue anchors on the second side (e.g., the left atrium side) of the septal wall. The delivery tubes 160, 162 can be positioned through the septal wall.

At block 418, the first and second needles 170, 172 are de-energized.

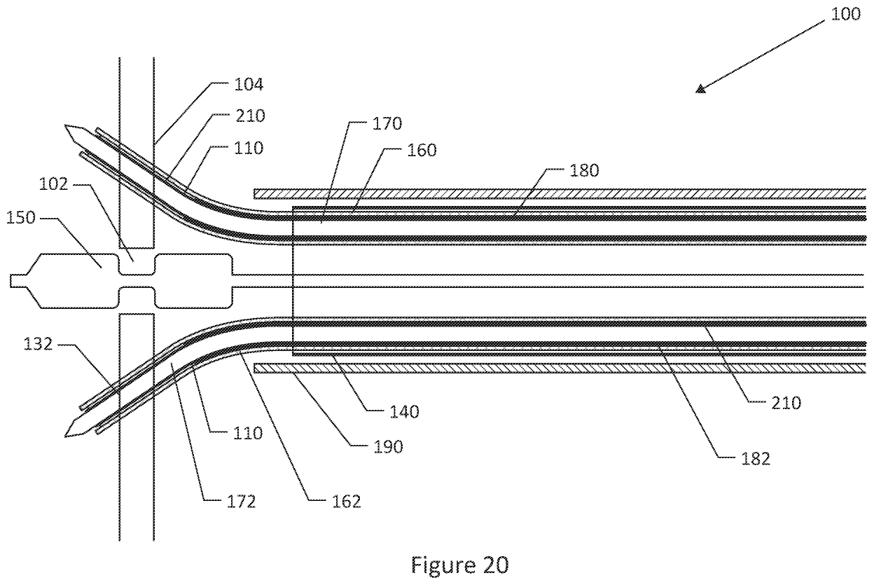

At block 420, the delivery tubes 160, 162 of the anchor catheter 140 can be deployed. In some examples, the actuating knob 144 of the anchor catheter 140 can be actuated to deploy the delivery tubes 160, 162 from the distal end of the anchor catheter 140. The delivery tubes 160, 162 can be deployed by movement of the delivery tubes 160, 162 relative to the delivery catheter 140, such that the delivery catheter 140 no longer constrains the delivery tubes 160, 162. As illustrated in FIG. 20, the delivery tubes 160, 162 when deployed can be advanced through the openings created by the first and second needles 170, 172. The delivery tubes 160, 162 can be advanced over the needles 170, 172, which are positioned through the septal wall. As illustrated in FIG. 6, the anchors 110 are located at each of the distal ends of the delivery tubes 160, 162. The suture 112 is secured to the tissue anchor 110. In the illustrated arrangement, the suture 112 is secured to the side of the tissue anchor 110. An end of the suture 112 can be received within an opening of the tissue anchor 110. The end of the suture 112 can be attached to portion of the anchor 110 or retained within an opening of the anchor 110 by any suitable arrangement, such as adhesives or mechanical fixation (e.g., a set screw). Other suitable arrangements are also possible. For example, the suture 112 can be secured to the body of the tissue anchor 110. As previously described and as illustrated in FIG. 6, the distal ends of the anchors 110 can extend beyond the distal end of the respective delivery tubes 160, 162, such that the distal ends of the anchors are located distally from the distal ends of the delivery tubes 160, 162. This creates a chamfered profile. The chamfered profile allows the tissue anchor 110 and delivery tubes 160, 162 to cooperate which facilitates the passing of the delivery tubes 160, 162 through the opening of the septal wall and over the needles 170, 172. Once in position, the delivery tubes 160, 162 can be positioned through the septal wall, such that the distal ends of the delivery tubes 160, 162 can be positioned are on the second side of the septal wall. The first side of the septal wall being the side at which the introducer sheath 190 is located, which can be the right atrium side of the septal wall. The second side being opposite the first side of the septal wall. The second side can be the left atrium side of the septal wall. The pushers 180, 182 can remain positioned proximally to the anchors 110. The pushers 180, 182 can hold the anchors 110 in place relative to the delivery tubes 160, 162.

In some examples, at blocks 416-420, each needle 170, 172 can be advanced together with the respective delivery tubes 160, 162 and the respective anchor 110 through the septal wall. In this manner, the first needle 170 cooperates with the first delivery tube 160 and first anchor 110 can form a chamfered surface or edge and can cooperate to together puncture through the septal wall. The second needles 172 can cooperate with the second delivery tube 162 and the second anchor 110 in a similar manner. As described above, the needles 170, 172 can be energized to further aid the passing of the needle 170, 172 through the septal wall.

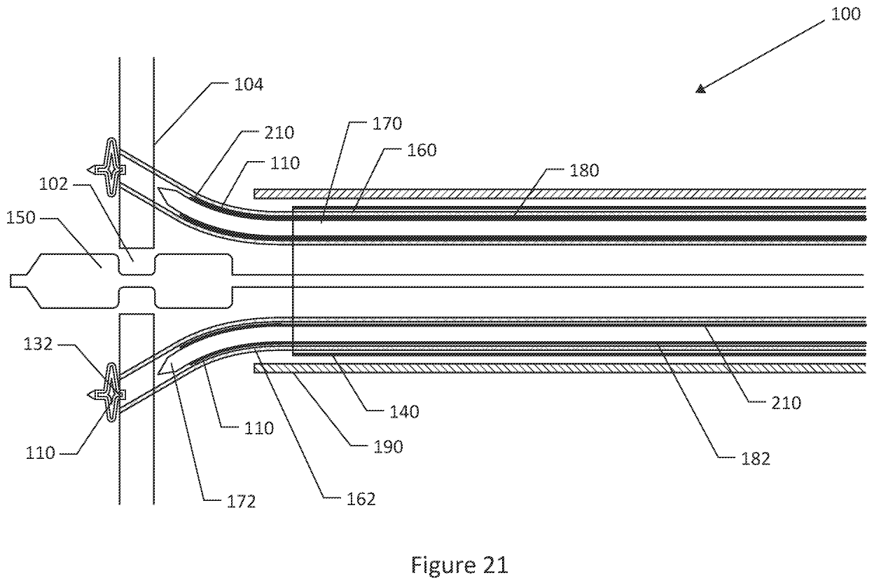

At block 422, the anchors 110 can be deployed such that the anchors 110 are positioned distal from the distal end of the delivery tubes 160, 162, such as illustrated in FIG. 21. The anchors 110 can be moved distally relative to the delivery tubes 160, 162. In some examples, the pushers 180, 182 can be advanced relative to the delivery tubes 160, 162 to deploy the anchors 110. As illustrated in FIG. 6, the pushers 180, 182 can be tubes that are positioned proximally from the anchors 110, within the delivery tube 160, 162, and can receive the needles 170, 172 therein. The pushers 180, 182 can be advanced distally within the delivery tubes 160, 162. This can in turn push the anchors 110 out of the respective distal ends of the delivery tubes 160, 162. As the distal ends of the delivery tubes 160, 162 are positioned on the second side of the septal wall (e.g. the left atrium side) when the anchors 110 are deployed from the delivery tubes 160, 162, the anchors 110 are then positioned on the second side (e.g., the left atrium side) of the septal wall, such as shown in FIG. 10D. The anchors 110 can be deployed from the delivery tubes 160, 162 such that they are no longer constrained by the delivery tubes 160, 162. When constrained by the delivery tubes 160, 162, the anchors 110 can be in a first configuration in which the anchors 110 are radially collapsed and elongate, such as shown in FIG. 10A. When no longer constrained by the delivery tubes 160, 162, the anchors 110 can transform from the first configuration to a second configuration. In the second configuration, the tissue anchors 110 can be radially expanded and shorter in length, such as shown in FIGS. 10B-10C. As described in FIGS. 10A-10E, the anchors 110 can be self-expanding.

At block 422, instead of the pushers 180, 182 being advanced relative to the delivery tubes 160, 162 to deploy the anchors 110, the delivery tubes 160, 162 can instead be withdrawn while the anchors 110 and pushers 180, 182 remain in place. The delivery tubes 160, 162 can initially be positioned through the septal wall such that the distal ends each extend beyond the septal wall by at least the length of the tissue anchor. This allows the anchors 110 positioned within the distal ends of the delivery tubes 160, 162 to be positioned at the desired location on the second side of the septal wall. The delivery tubes 160, 162 can then be withdrawn relative to the anchors 110, such that the anchors 110 are deployed from the respective delivery tubes 160, 162. In some examples, the anchors 110 can be held in place relative to the delivery tubes 160, 162 by the pushers 180, 182. The pushers 180, 182 can hold the anchors 110 in position as the delivery tubes 160, 162 are withdrawn.

At block 424, the pushers 180, 182, the needles 170, 172 and the delivery tubes 160, 162 can be retracted. In some examples, the pushers 180, 182, needles 170, 172, and the tubes 160, 162 can be each fully retracted within the anchor catheter 140.

At block 426, the balloon member 150 can be deflated. In some examples, the deflated balloon member 150 can remain outside the anchor catheter 140. In some examples, the deflated balloon member 150 can be retracted within the anchor catheter 140.

At block 428, the anchor catheter 140 and the deflated balloon member 150 can be withdrawn. The withdrawn anchor catheter 140 can retain at least the retracted pushers 180, 182, needles 170, 172, and delivery tubes 160, 162. The anchors 110 can remain implanted in the suture wall after withdrawal of the anchor catheter 140 and the deflated balloon member 150, as illustrated in FIG. 10D. As previously described, each of the anchors 110 can have one or more sutures 112 attached thereto. The one or more suture 112 can connect the anchors 110 to one another.

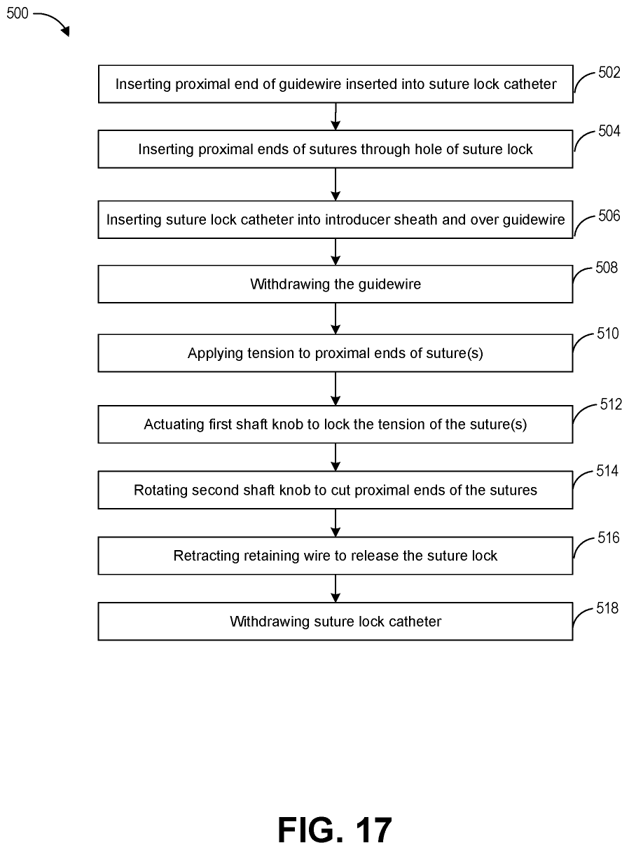

FIG. 17 is a flow diagram illustrating a method 500 of applying a suture lock to the one or more sutures of the tissue anchors. The method of FIG. 17 can be a continuation of the method of FIG. 16 as a method 400 of repairing a tissue defect. In other examples, other methods of fixing the effective length of the one or more sutures 112 connecting the anchors 110 can be used.

The introducer sheath 190 can be previously placed through the femoral vein into the heart and positioned adjacent the septal defect 102. The guidewire 152 can be previously in place through the introducer sheath 190 and through the septal defect 102. In some examples, the introducer sheath 190 and the guidewire 152 can remain in place at the end of method 400.

At block 502, the proximal end of the guidewire 152 can be positioned into a suture lock catheter 270. In some examples, the proximal end of the guidewire can be inserted into the suture lock catheter 270.

At block 504, the proximal ends of the one or more sutures 112 can be positioned or threaded through a cross drilled hole or passage 222 of the suture lock 114, as illustrated in FIG. 8A.

At block 506, the suture lock catheter 270 can be advanced over the guidewire 152 and within the introducer sheath 190. Similar to the step at block 408, a peel-away sheath can optionally be used to facilitate entry of the suture lock catheter 270 into the introducer sheath 190. An example of the suture lock catheter 270 is illustrated in FIGS. 9A-9B. As illustrated in FIG. 9B, a suture lock and cutter 114 can be placed at a distal end of the suture lock catheter 270. The suture lock and cutter 114 can be retained in place at the distal end of the suture lock catheter 270 with a retaining wire 116 positioned between the body 220 of the suture lock 114 and the interior surface of the suture lock catheter 270. The suture lock catheter 270 can be advanced over the guidewire 152 and within the introducer sheath 190 to reach the desired location. In some examples, this desired location is the first side of the septal wall, which can be the right atrium side of the septal wall.

At block 508, guidewire 152 can be withdrawn.

At block 510, tension can be applied to proximal ends of the sutures 112. As tension is applied to the suture(s) 112, the effective length of the suture(s) 112 between the tissue anchors 110 can be decreased and the anchors 110 can be drawn together. As the anchors 110 are drawn together, the tissue defect 102 can be closed.

At block 512, the tension of the suture(s) can be locked. In some examples, the first drive shaft 272 of the suture lock catheter 270 can be actuated to lock the suture lock 114, which can in turn lock the tension of the suture(s) 112. In some examples, the first drive shaft 272 can be actuated by rotation. The actuation of the first drive shaft 272 can move a first driver 240. The first driver 240 can in turn move a locking screw 224. The locking screw 224 can have a smooth, flat bottom, such as shown in FIG. 8B. When the locking screw 224 is advanced into the passage 222 by the first driver 240, the end of the locking screw 224 can capture a portion of the suture(s) 112 against an opposite surface of the passage 222 to secure the suture(s) 112 and fix the tension of the suture(s) 112.

At block 514, proximal ends of the suture(s) can be cut. In some examples, the second drive shaft 274 of the suture lock catheter 270 can be actuated to actuate the suture lock 114, which can in turn cut the proximal ends of the suture(s) 112. In some examples, the second drive shaft 274 can be actuated by rotation. The actuation of the second shaft driver 274 can move a second driver 240. The second driver 240 can in turn move a cutting screw 230. The cutting screw 230 can have a sharpened end such as shown in FIG. 8B. When the cutting screw 230 is advanced into the passage 222 by the first driver 240, the end of the locking screw 224 can cut the suture(s) 112 against an opposite surface of the passage 222.

At block 516, the suture lock can be released from the suture lock catheter. In some examples, the retaining wire 116 can be retracted to release the suture lock 114 from the suture lock catheter 270.

At block 518, the suture lock catheter 270 can be withdrawn.

### CONCLUSION

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

1. A system for repairing a tissue defect, the system comprising:
a locating member configured to extend into the tissue defect and to locate one or more components of the system relative to the tissue defect, wherein the locating member defines a non-circular outside shape.

2. The system for repairing a tissue defect of claim 1, wherein the locating member comprises a non-circular balloon.

3. (canceled)

4. (canceled)

5. (canceled)

6. The system for repairing a tissue defect of claim 1, wherein the locating member comprises an expandable body, such as a cage or trusswork.

7. (canceled)

8. (canceled)

9. The system for repairing a tissue defect of claim 1, further comprising a first tissue anchor configured to be implanted into tissue at a first location, and a second tissue anchor configured to be implanted into tissue at a second location.

10. (canceled)

11. The system for repairing a tissue defect of claim 9, wherein the non-circular outside shape of the locating member defines a major axis, and wherein the first tissue anchor and the second tissue anchor are equally spaced on opposite sides of the major axis.

12. The system for repairing a tissue defect of claim 11, wherein the non-circular outside shape of the locating member defines a minor axis, and wherein the first tissue anchor and the second tissue anchor are located on the minor axis.

13.-34. (canceled)

35. A system for repairing an atrial septal defect, the system comprising:
a catheter;
a centering member slidably supported within the catheter or beyond a distal end of the catheter, wherein the centering member is configured to extend from the catheter into the atrial septal defect;
a first delivery tube slidably supported within the catheter;
a first tissue anchor disposed within a distal end of the first delivery tube, wherein the first delivery tube is configured to be advanced from the catheter to allow implantation of the first tissue anchor into tissue at a first location adjacent a first side of the atrial septal defect;
a second delivery tube slidably supported within the catheter; and
a second tissue anchor disposed within a distal end of the second delivery tube, wherein the second delivery tube is configured to be advanced from the catheter to allow implantation of the second tissue anchor into tissue at a second location adjacent a second side of the atrial septal defect.

36.-61. (canceled)

62. A system for repairing an atrial septal defect, the system comprising:
a centering member configured to extend into the atrial septal defect, wherein the centering member defines a non-circular outside shape;
a first tissue anchor configured to be implanted into tissue at a first location adjacent a first side of the atrial septal defect; and
a second tissue anchor configured to be implanted into tissue at a second location adjacent a second side of the atrial septal defect.

63. (canceled)

64. The system of claim 62, wherein the centering member is configured to extend into the atrial septal defect while in a collapsed introduction configuration and expand into an expanded deployed configuration after being positioned within the atrial septal defect.

65.-79. (canceled)

80. The system of claim 62, further comprising a suture coupled to at least one of the first tissue anchor and the second tissue anchor.

81. The system of claim 62, further comprising a suture coupled to each of the first tissue anchor and the second tissue anchor.

82. The system of claim 81, further comprising a suture lock configured to secure a length of a tensioned portion of the suture between the first tissue anchor and the second tissue anchor.

83. The system of claim 72, further comprising a suture cutter.

84. A system for repairing an elongated atrial septal defect, the system comprising:
a centering member configured to extend into the elongated atrial septal defect, wherein the centering member is configured to conform to a shape of the elongated atrial septal defect and to orient one or more components of the system with a lengthwise direction of the elongated atrial septal defect.

85. (canceled)

86. (canceled)

87. A system for repairing an atrial septal defect, the system comprising:
a catheter;
a centering member slidably supported within the catheter or beyond a distal end of the catheter, wherein the centering member is configured to extend from the catheter into the atrial septal defect;
a first tissue anchor carried by the catheter and configured to be advanced from the catheter to a first location within 3-5 mm of a first side of the atrial septal defect for implantation into tissue;
a second tissue anchor carried by the catheter and configured to be advanced from the catheter to a second location within 3-5 mm of a second side of the atrial septal defect for implantation into tissue.

88. (canceled)

89. (canceled)

90. (canceled)

91. (canceled)

92. A method of repairing a tissue defect, the method comprising:
positioning an introducer sheath adjacent a defect of a tissue wall on a first side of the tissue wall;
positioning a guidewire through the defect of the tissue wall;
inserting a proximal end of the guidewire into a centering balloon lumen;
advancing an anchor catheter over the guidewire and within the introducer sheath;
advancing a balloon member through the anchor catheter to position the balloon member partially through the defect;
inflating the balloon member;
advancing a first needle of the anchor catheter through a tissue wall at a first location of the tissue wall, wherein the first location is adjacent to the defect;
deploying a first delivery tube of the anchor catheter through a first hole created by the first needle; and
deploying a first tissue anchor from the first delivery tube to a second side of the tissue wall.

93. The method of claim 92, wherein prior to advancing the first needle through the tissue wall needle at the first location, the method comprises deploying the first delivery tube of the anchor catheter and the second delivery tube of the anchor catheter adjacent to the first location of the tissue wall on the first side of the tissue wall, wherein the first delivery tube and the second delivery tube are each configured to curve radially outward from the delivery catheter.

94. The method of claim 92, further comprising energizing the first needle prior to advancing the first needle through the tissue wall.

95. The method of claim 92, wherein advancing the first needle of the anchor catheter through the tissue wall at the first location comprises advancing each of the first delivery tube and the first tissue anchor through the tissue wall with the first needle, wherein distal ends of the first needle, the first delivery tube, and the first tissue anchor form a chamfered edge.

96. (canceled)

97. (canceled)

98. (canceled)

99. (canceled)

100. (canceled)

101. A system for repairing a tissue defect, the system comprising:
a locating member configured to extend into the tissue defect and to locate one or more components of the system relative to the tissue defect, wherein the locating member defines a circular outside shape.

102. (canceled)

103. (canceled)

104. (canceled)

105. (canceled)

106. (canceled)
